(19) United States
(12) Patent Application Publication
(10) Pub. No.: US 2008/0255476 A1
(43) Pub. Date: Oct. 16, 2008

(54) Methods and devices for treating obesity

(76) Inventors: Tom Boyajian, (US); Greg Lambrecht, (US)

Correspondence Address: Tom Boyajian, 1 Kilby Street, Wilmington, MA, 01887, US

(21) Appl. No.: 11/983,905

(22) Filed: Nov. 13, 2007

Related U.S. Application Data
(60) Provisional application No. 60/860,119, filed on Nov. 20, 2006.

(51) Int. Cl. A61F 2/04; A61N 5/067; A61B 5/107

(52) U.S. Cl. 600/593; 607/89; 623/23.65; 600/587

(57) ABSTRACT

Methods and devices for treating obesity are provided. The consumption of calorie dense, lipid rich, or fatty foods is discouraged through the modulation of a subjects gallbladder function or output. Disclosed are devices and methods for delivering devices within the gallbladder and associated ducts and vasculature; other methods involve implanting devices on or around the gallbladder and associated ducts and vasculature. Further treatments involve the use of energy, surgery, or chemicals to alter the function of the gallbladder and biliary system.

### CROSS-REFERENCE TO RELATED APPLICATIONS

This application claims the benefit of U.S. Provisional Application No. 60/860,119 filed Nov. 20, 2006 herein incorporated by reference.

### BACKGROUND OF THE INVENTION

1. Field of the Invention

The invention relates generally to treating, reversing, or preventing obesity. More specifically it relates to permanently, temporarily or reversibly altering an aspect or function of the gallbladder or portions of the biliary system to discourage a person from eating certain types and amounts of foods.

2. Description of the Related Art

Obesity is a pathological condition arising from too much body fat and is measured according to a body mass index.

According to the world health organization obesity has reached epidemic proportions globally, with more than 1 billion adults overweight—at least 300 million of them clinically obese and is a major contributor to the global burden of chronic disease and disability. Increased consumption of more energy-dense, nutrient-poor foods with high levels of sugar and saturated fats, combined with reduced physical activity, have led to obesity rates that have risen three-fold or more since 1980.

Obesity and being overweight pose a major risk for serious diet-related chronic diseases, including type 2 diabetes, cardiovascular disease, hypertension and stroke, and certain forms of cancer. The health consequences range from increased risk of premature death, to serious chronic conditions that reduce the overall quality of life. Of especial concern is the increasing incidence of child obesity.

Currently treatments for obesity include diet, exercise programs, drugs such as sibutramine and orlistat, and surgical interventions such as gastric bypasses. Vertical banded gastroplasty involves dividing the stomach into two parts with staples and inserting a band to limit the stretch in the opening between the two sections. Risks include wearing away of the band and breakdown of the staple line. In a small number of cases, stomach juices may leak into the abdomen or infection or death from complications may occur. In a laparoscopic gastric banding procedure an inflatable band is placed around the upper stomach to create a small pouch and narrow passage into the remainder of the stomach. This limits food consumption and creates an earlier feeling of fullness. Complications may include nausea and vomiting, heartburn, abdominal pain, band slippage, or pouch enlargement. Another procedure, roux-en-Y gastric bypass involves making the stomach smaller by using surgical staples to create a small stomach pouch. The pouch is attached to the middle part of a small intestine. Food bypasses the upper part of the small intestine and stomach and goes into the middle part of the small intestine through a small opening. Bypassing the stomach limits the amount of food a person can eat. By bypassing part of the intestine, the amount of calories and nutrients the body absorbs is reduced. One risk for patients is “dumping syndrome.” This happens when the stomach contents move too rapidly through the small intestine. Symptoms may include nausea, weakness, sweating, faintness, and diarrhea after eating. Side effects include infection, leaking, pulmonary embolism (sudden blockage in a lung artery), gallstones, and nutritional deficiency. Finally, a biliopancreatic diversion (BPD) involves removing a large part of the stomach. The amount of food is restricted, in addition to stomach acid production. The small pouch that remains is connected directly to the final segment of the small intestine, completely bypassing other parts of the small intestine. A common channel remains in which bile and pancreatic digestive juices mix prior to entering the colon. Weight loss occurs since most of the calories and nutrients are routed into the colon where they are not absorbed. This procedure is less frequently used than other types of surgery because of the high risk for nutritional deficiencies.

It is estimated that 40% of gastric bypass patients develop complications following their surgery. Leaks, infection and respiratory failure are among the most common serious complications. Thus, there is a need for a surgical alternative that is less invasive, has less complications, does not interfere with nutrient absorption, and prevents relapses.

### SUMMARY OF THE INVENTION

According to one or more methods described herein the consumption of calories dense, lipid rich, or fatty foods is discouraged through the modulation of gallbladder function or output. Disclosed are devices and methods for delivering devices within the gallbladder and associated ducts and vasculature; other methods involve implanting devices on or around the gallbladder and associated ducts and vasculature. Further treatments involve the use of energy, surgery, or chemicals to alter the function of the gallbladder and biliary system.

In an exemplary embodiment a method of treating obesity involves permanently or temporarily lowering one or more of the following aspects of a subjects gallbladder: motility, evacuation fraction, turnover rate, bile flow rate; wherein said lowered aspect causes the consumption of certain amounts and types of food to result in physical discomfort in the subject. Other aspects of the invention can involve one or more of the following: the application energy to a portion of the gallbladder or cystic duct, causing a stricture or stenosis in at least a porting of the gallbladder or cystic duct; inserting an implant within the gallbladder or cystic duct wherein said device at least partially limits the flow of bile or interferes with the contraction of the gallbladder; surgically or chemically altering or damaging the tissue of the gallbladder or cystic duct; causing neuropathy or paralyzing a portion of the cystic duct; diminishing the capacity of duodenum to deliver CCK or inhibiting the capacity of the gallbladder to absorb CCK; pacing at least a portion of the biliary system, causing neuropathy of or paralyzing a portion of the gallbladder and monitoring an aspect of the biliary system.

### BRIEF DESCRIPTION OF THE DRAWINGS

FIG. 1 is a simplified frontal view of the gallbladder and a portion of the digestive system. Also shown is constricting band around the gallbladder

FIG. 2 is frontal view of a portion of the digestive system and a band device around the cystic duct.

FIG. 3 is frontal view of a portion of the digestive system and a mesh device around the gallbladder.

FIG. 4 is frontal view of a portion of the digestive system and shows a partially occlusive clip around cystic duct.

FIG. 5 is frontal view of a portion of the digestive system and shows a suture or staple through the gallbladder wall in one or more places and constricted or tightened to reduce gallbladder volume or restrict expansion.

FIG. 6 is frontal view of a portion of the digestive system and shows an endoluminal stent or tube placed in the cystic duct to restricting, reducing, or modulating flow of bile.

FIG. 7 is frontal view of a portion of the digestive system and shows a flow restricting valve placed in the cystic duct. This valve acts to restrict inflow of bile, but could further reduce flow out of the gallbladder.

FIG. 8 is frontal view of a portion of the digestive system and shows an occlusive plug within the cystic duct. Such a plug could be formed in situ using glues, etc.

FIG. 9 is frontal view of a portion of the digestive system and shows the application of radio-frequency energy through an RF or other energy form delivering grasper placed around cystic duct either through open surgery or laparoscopy.

FIG. 10 is frontal view of a portion of the digestive system and shows a radio-frequency or other energy delivering catheter placed endoluminally within the cystic duct to apply heat and cause scarring and/or stricture of the duct. Temperature measuring could be employed to optimize the thermal dose delivery.

FIG. 11 is frontal view of a portion of the digestive system and shows artificial gallstones placed within the gallbladder either endoluminally or through the gallbladder wall.

FIG. 12 is frontal view of a portion of the digestive system and shows the injection of flowable substance into the gallbladder wall. The substance could be used to bulk the wall to cause reduction of gallbladder volume, or could be used to cause a reduction in the muscular activity of the wall.

FIG. 13 is frontal view of a portion of the digestive system and shows embolization of the cystic artery to cause a reduction or elimination of flow to at least a portion of the gallbladder. Alternatively, distal branches could be selectively embolized.

FIG. 14 is frontal view of a portion of the digestive system and shows electrical pacing of the celiac (caeliac) plexus to cause a change in the activity of the gallbladder. Pacer is shown in the chest wall with a lead wire running to a pacing lead in contact with the celiac plexus.

### DETAILED DESCRIPTION

The biliary system is comprised of the gallbladder and the ducts that carry bile and other digestive enzymes from the liver, gallbladder, and pancreas to the small intestine. The gallbladder is about 10-12 cm long in humans. It is connected to the liver and the duodenum by the biliary tract which includes the cystic duct and common bile duct. The cystic duct terminates in a series of spiral valves and connects the gallbladder to the common hepatic duct to form the common bile duct. The common bile duct then joins the pancreatic duct, and enters through the hepatopancreatic ampulla at the major duodenal papilla.

The gallbladder has a simple columnar epithelial lining characterized by recesses called Aschoff's recesses, which are pouches inside the lining. Under the epithelium there is a layer of connective tissue (lamina propria). Beneath the connective tissue is a wall of smooth muscle (muscularis muscosa) that contracts in response to cholecystokinin, a peptide hormone secreted by the duodenum. There is essentially no submucosa separating the connective tissue from serosa and adventitia.

The gallbladder stores about 50 ml of bile (1.7 US fluid ounces/1.8 Imperial fluid ounces), which is released when food containing fat enters the digestive tract, stimulating the secretion of cholecystokinin (CCK). The bile, produced in the liver, emulsifies fats and neutralizes acids in partly digested food. After being stored in the gallbladder, the bile becomes more concentrated than when it left the liver, increasing its potency and intensifying its effect on fats. Most digestion occurs in the duodenum.

Patients with either a dysfunctional gallbladder or one that is at least partially obstructed by gall stones or stricture feel pain when consuming foods that cause the gallbladder to expulse bile through the gallbladder or cystic duct into the digestive tract. This pain acts as an effective deterrent to the consumption of large quantities of food and/or the consumption of high caloric foods, particularly those containing fat. Such patients often experience a significant decrease in weight as a consequence of the resultant highly restricted diet. However, unlike patients with a dysfunctional stomach or intestine, the patient is still able to absorb essential nutrients from the food that is ingested.

Rather than surgically altering the stomach which poses significant life threatening complications, has a poor success rate, and diminishes the capacity of the organ to absorb required nutrients, the current invention is directed at altering, modulating, or limiting the function, mechanics, or output of the gallbladder to discourage a subject from eating foods high in fat or otherwise rich in calories known for causing obesity, as well as discouraging high volumes of food which can also cause discomfort.

Another method according to one or more aspects of the invention involves intentionally inducing an obstructed or dysfunctional gallbladder in obese patients with the result that the patient is deterred from eating high caloric or high volume diets, thus inducing weight loss. In the context of this patent “inducing a dysfunctional gallbladder” shall mean a gallbladder or its associated tissues and ducts with diminished or simply lowered motility, evacuation fraction, turnover rate, or bile flow rate.

At least partial obstruction of the flow of bile from the gallbladder can be accomplished by creating a stricture or stenosis of the outflow from the gallbladder. This can be accomplished either from the outside of the gallbladder or cystic duct or from the inside.

From an external perspective of the biliary system, a stenosis or stricture can be achieved with a circumferential band or mesh around either the gallbladder or cystic duct. The band could be inflatable or adjustable to better control the compression of the gallbladder or cystic duct either intra or post-operatively. Inflation can be achieved with a source or gas or liquid such as saline. Different levels of inflation can be used to affect varied levels of constriction and can be varied over time or even remotely. The inflation pressure can be monitored and tuned to a specific level to achieve a desired flow restriction pressure. Alternatively, a partially-occluding clip could be placed. Flow rate through the cystic duct or the pressure necessary to drive flow may be measured in combination with the compression of the gallbladder or cystic duct to achieve a desired resultant flow or pressure. Pressure or flow rate may be measured through either direct sensing of fluids within the flow path or by observing the flow remotely such as with the x-ray or fluoroscopic observation of the flow of radio-opaque fluid through the gallbladder or duct.

In another embodiment, a suture or staple 15 is placed through the wall of the gallbladder and then constricted bringing portions of the gallbladder wall closer together thereby reducing the internal volume of the gallbladder. The clip, band, mesh, suture or staple can be either elastic or rigid. In an elastic embodiment, the elasticity would preferably be tuned to allow bile flow at a certain, elevated pressure. In a rigid clip embodiment, the device would be sized to achieve a reduction in size of the flow-path for bile into, through, or out of either the gallbladder or cystic duct. Either approach could be accomplished using either a temporary or permanent implant. A temporary implant could degrade over time. Such bands, clips, sutures, staples, or meshes could be placed using standard open surgical techniques, endoscopic, and laparoscopic techniques, or through other, minimally invasive approaches to the gallbladder common in the art.

Alternatively, energy could be applied to all or part of the gallbladder or cystic duct to induce injury that damages tissue (including nerves) and/or results in stricture either directly or via a healing response. Energy could be delivered using RF (either mono or bi-polar), microwave, thermal, or laser. In addition, cryotherapy could be applied to induce damage or stricture, again from a healing response. Stricture or a reduction in the size of the gallbladder or cystic duct could be done to restrict bile inflow to or outflow from the gallbladder. Local temperature could be measured to control the extent or nature of the tissue damage resulting from such thermal treatments of the gallbladder or cystic duct.

According to one or more aspects of the invention, the output of the gallbladder can also be altered, controlled, modulated through the implantation of an implant within the interior of the gallbladder and associated ducts and vasculature. Obstruction of the flow-path could be achieved using an implant such as tube or stent that is placed in the cystic duct, the neck of the gallbladder or within the gallbladder itself. By occupying space within the flow-path for bile, such an implant would restrict flow. The implant could also be used to create an injury and healing response to result in a partially scarred-down cystic duct or neck of the gallbladder. The tube, stent or implant could be of a fixed diameter, self expanding, or expandable through plastic deformation (i.e. balloon expandable). The stent, tube or implant, could also be either permanent, removable/retrievable or degrade over time.

In an alternative embodiment, partial obstruction of the gallbladder could be achieved by inserting one or more temporary or permanent gallstone-like objects into the gallbladder. Such stone implants could be made from metals, plastics, hydrogels, and the like. Alternatively, the artificial stones could be made from calcium compounds, salts, collagen, cellulite, cholesterol, lecithin, acids or other degradable or non-degradable biomaterial. The size and quantity of the stones could be optimized for each patient to be large enough to obstruct the cystic duct without obstructing the hepatic or common bile ducts once passed. Any one or a number of the stones could be inflatable or swell to achieve an optimal size following placement.

In an alternative embodiment, the ejection fraction of the gallbladder could be reduced by creating a physical obstruction to the full contraction of the gallbladder wall during expulsion of bile. The obstruction could involve an inflatable object or balloon within the gallbladder. Alternatively, a metallic or polymeric frame could be placed within the gallbladder or expanded therein. Such a frame or balloon could contact the inner wall of the gallbladder during constriction and prevent or resist the full contraction of the gallbladder, limiting or reducing outflow without creating a physical obstruction to the flow itself. Outflow of bile could be limited to either a specific volume or a fraction of the patients own ejection fraction. For example, the ejected volume of bile could be reduced from 50 ml to 20 ml. Alternatively, the ejection fraction could be reduced from 80% to 20%.

In another aspect of one or more embodiments of the invention a valve implant could be placed into the neck of the gallbladder or into the cystic duct. Such a valve could provide a specific or variable resistance to flow. It could allow or restrict flow in either one or both directions through the valve.

An aspect of the inventive method can alternatively involve the partial or total obstruction of the gallbladder and associated tissues, ducts, and vasculature by endoluminal application of energy such as heat, RF, microwave or laser. Temperature control during the application of energy could be used to deliver a specific thermal dosing profile to achieve a specific tissue response (such as stricture or stenosis without ablation).

In another aspect of one or more aspects of the invention, a treatment can involve the injection of an augmenting material into the wall of the gallbladder or cystic duct. This could be continued until a desired luminal or gallbladder volume restriction was achieved. The desired restriction could be determined by measuring flow rate or pressure to drive flow through the gallbladder or cystic duct. Alternatively, the level of volume reduction in the gallbladder could be observed during material injection. Such a material could also be injected to form a partial or total occlusion of the cystic duct. It could alternatively be injected into the gallbladder and fill a specified volume of the gallbladder reducing its filling or ejection capacity. Such a material could be permanent or could degrade over time. Materials could be adhesive, such as glues (fibrin glue, etc.), natural or synthetic polymers, metals, or ceramics. The injectable materials could be solids, fluids, or could be phase changing from fluid to solid as a result of temperature (cooling or heating to body temperature) or chemical reaction.

Any of the above techniques involving the creation of an obstruction can additionally involve use of monitoring of one or more characteristics of the flow of bile into, through and/or out of the gallbladder. Pressure, volume, and/or flow rate could readily be monitored using a variety of available devices. Dye could also be injected through the gallbladder to visually monitor flow either directly or using intra-operative radiography. Intravenous dye could also be used, such as in HIDA scanning or cholescintigraphy.

In a further embodiment the gallbladder can alternatively be rendered partially or totally dysfunctional. This treatment could be preferably reversible, temporary, or permanent. Tissue modification, damage or neuropathy could be targeted at the entirety or a portion of the gallbladder and ducts or nerves. The modifications could be achieved surgically to alter its contractility or through pacing or destruction the nerves leading to the gall bladder.

Damage or altering the tissue mechanics of the gallbladder could be achieved through the application of energy, by the injection of chemical agents such as alcohol or botulism toxin, or by cryotherapy. Energy could be applied to the wall of the gallbladder or nerves using heat, radio frequency (RF), microwave, or laser. Single or multiple RF probes could be applied. Temperature monitoring during the application of heat or the use of cryotherapy could be performed to deliver specific thermal dosing profiles to the affected tissue.

Dysfunction could also be achieved by decreasing or eliminating blood flow to the gallbladder by restricting or eliminating flow through the cystic artery. Alternatively, flow through the cystic vein could be restricted or eliminated to create edema in the gallbladder.

Torsion of the gallbladder or cystic duct can be performed to affect a desired degree of constriction. The torsion can restrict blood flow or throughput of either or both on the gallbladder and cystic duct. The gallbladder or cystic duct and then be anchored or held in place with means know in the art such as sutures or glue. A device for applying torsional tension can be fashioned from a mesh, band, suture and means for anchoring or adhering to the tissue of the gallbladder or cystic duct.

Pacing of the gallbladder could be achieved by applying implantable electrodes to the portions of the celiac plexus or vagus nerve that innervate the gallbladder or to the intrinsic neurons of the gallbladder itself. Such pacing could be done to reduce or interrupt, inhibit, or alter the contraction of the gallbladder thereby inducing dysfunction.

Dysfunction, modulation, or alerting the physiology or biomechanics of the gallbladder could also be achieved through pharmaceutical means. It is known that the cholecystokinin (CCK) is involved in controlling gallbladder contractile activity. CCK inhibitors could be used to disrupt gallbladder function through pharmaceutical means. Alternatively the CCKA receptor could be blocked, preventing CCK activation either locally or systemically. CCK could also be continually administered to diminish the naturally occurring CCK production or efficacy. Implantation or delivery sites on and within organs of the biliary system can be utilized in delivering such pharmaceutical agents.

The chemicals and pharmaceutical means listed herein could be delivered or injected through means know in art. Moreover the implants such as bands, stents, tubes, synthetic gallstones and patches described herein could also be used to deliver such agents. Such devices could be simply implanted within the organs or adjacent and external or internal surface of the gallbladder and associated ducts, tissues and vasculature.

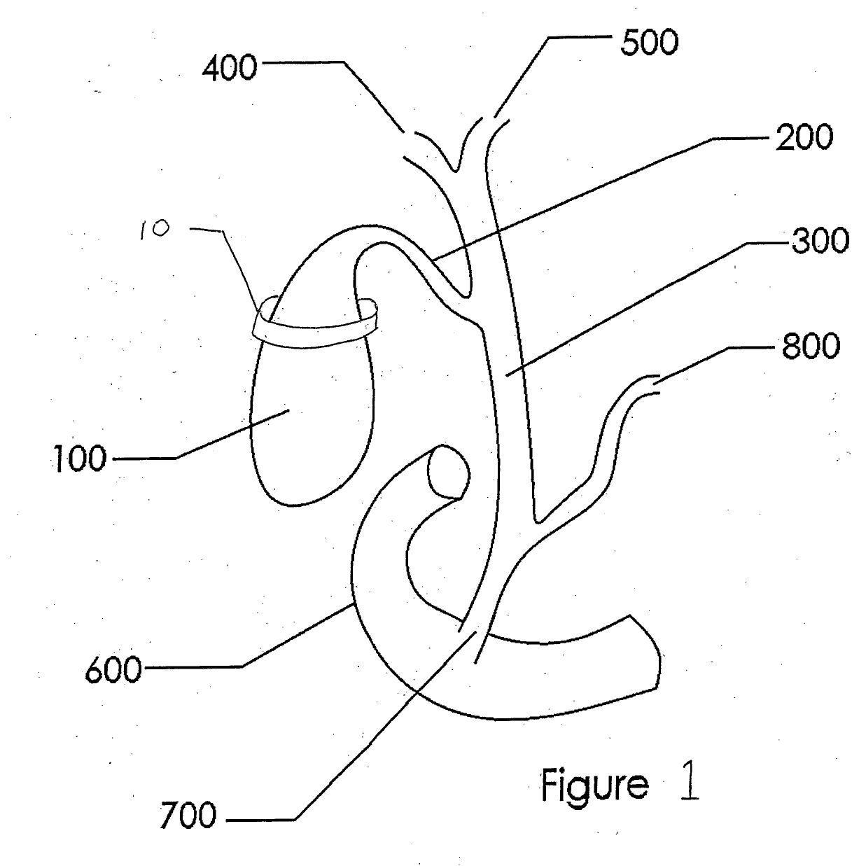

Based on the preceding discussion, the following examples and figures shall be used to illustrate certain features of one or more aspects of the invention. FIGS. 1-4 depict certain embodiments that exploit the outer surface of organs and tissues of the biliary system. FIG. 1 shows simplified frontal view of the biliary system and a portion of the digestive system. Shown are the left 400 and right 500 hepatic bile ducts that connect to the liver, common bile duct 300, cystic duct 200, gallbladder 100, duodenum 600, common outlet 700 and pancreas. Also shown is constricting band 10,20, ring 10,20, or suture 15 around the gallbladder 100 situated generally about its midsection.

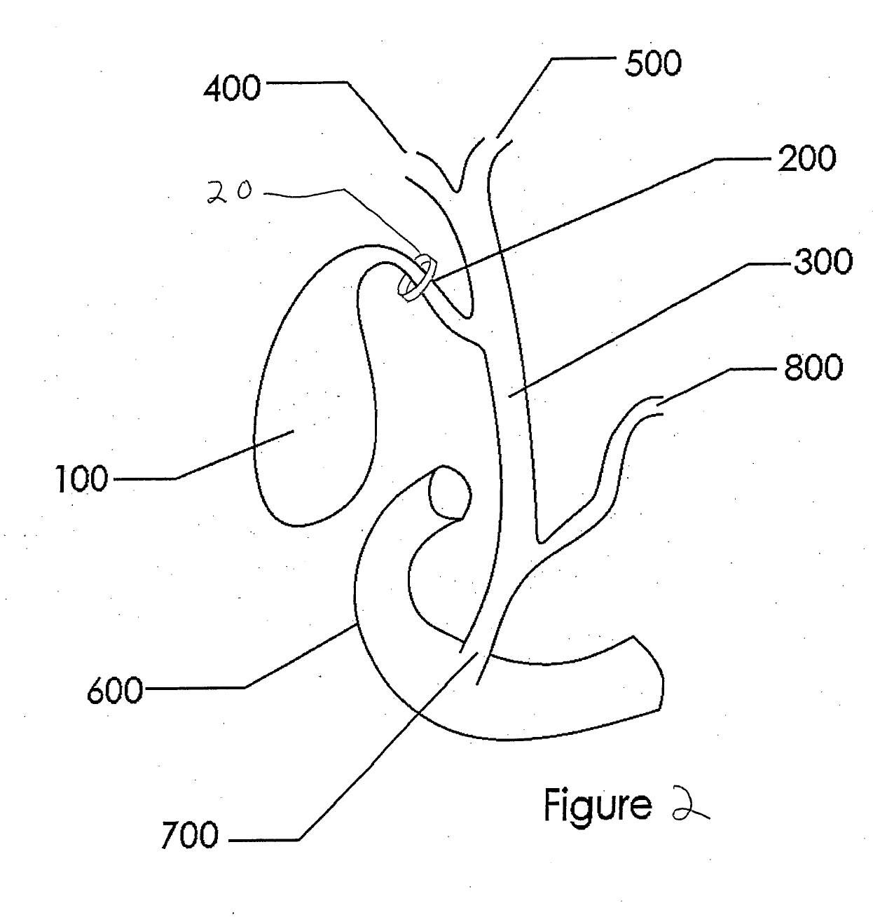

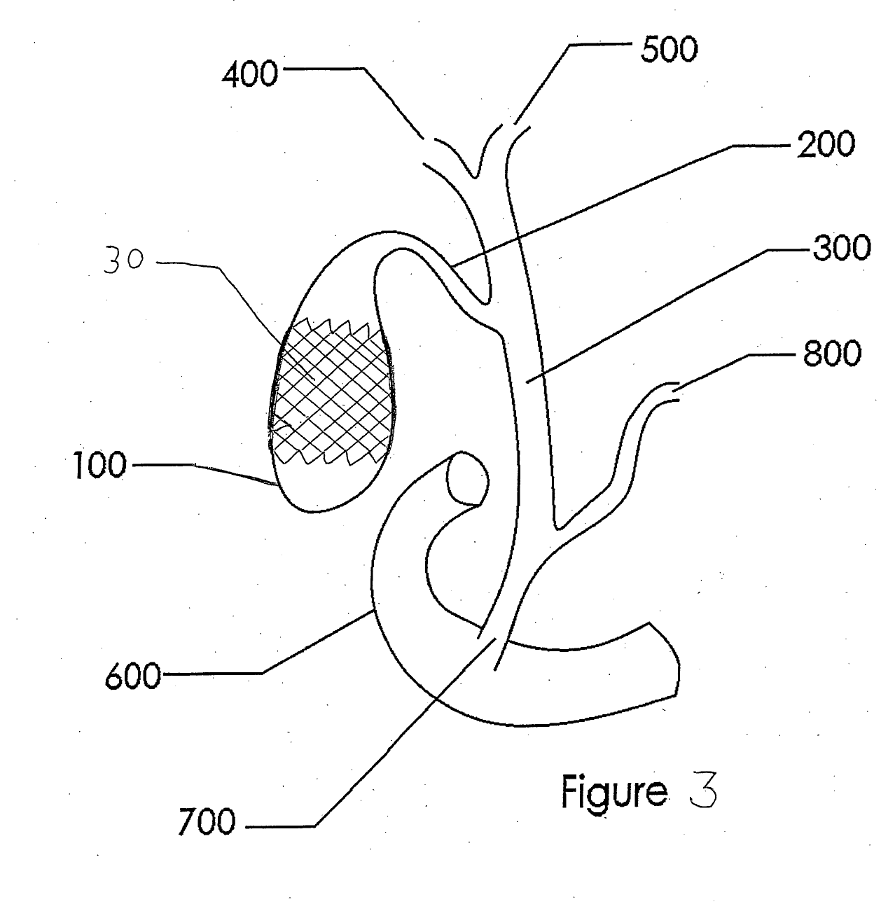

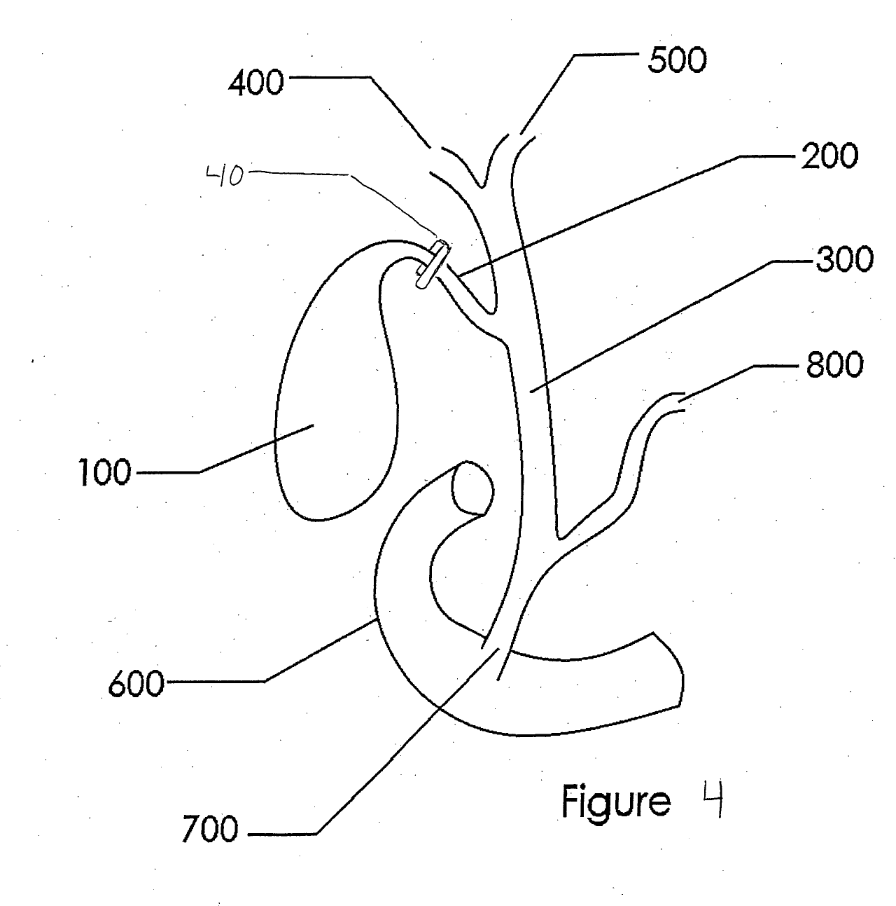

FIG. 2 shows an alternative placement of a band 10, 20, ring 10, 20, or clip 40 on or around the cystic duct 200. Though the device depicted in FIG. 2 is not shown actually contacting or constricting the duct this is for illustrative purposes and in use the device contacts the tissue. The device can be placed adjacent the common bile duct 300 or more towards the gallbladder 100. FIG. 3 shows a cylindrical mesh 30 covering a greater extent of the gallbladder. The mesh can function to constrict, compress or prevent the gallbladder 100 from expanding. Alternatively, the mesh 30 can be formed into a bag covering the entirety of the organ. FIG. 4 shows a clip 40 situated about the cystic duct 200. The clip 40 device only partially occludes the duct 200. A similar clip-like device for partial occlusion of the gallbladder 100 could also be employed.

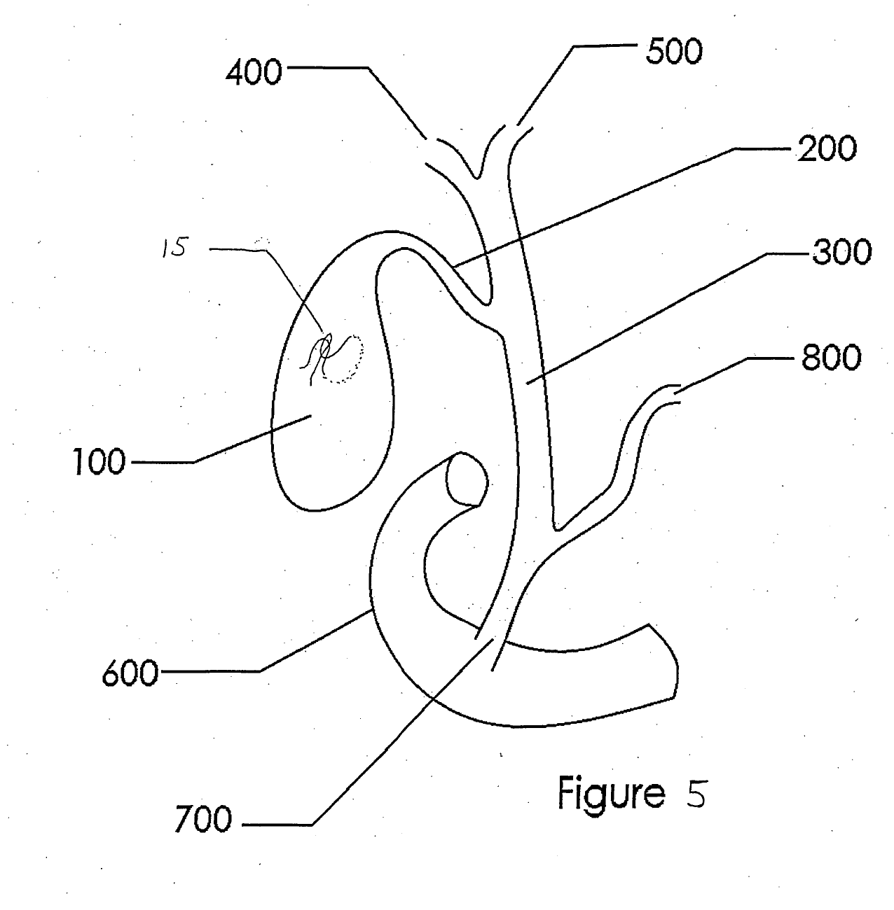

In another aspect of one or more embodiments of the invention, the gallbladder 100 and associated ducts and tissues can be modified with sutures 15, staples 15, or adhesives to reduce gallbladder 100 volume or restrict expansion. In FIG. 5, a frontal view of a portion of the digestive system is presented and shows a suture 15 or staple 15 through the gallbladder 100 wall in one or more places and constricted or tightened to reduce gallbladder 100 volume or restrict expansion.

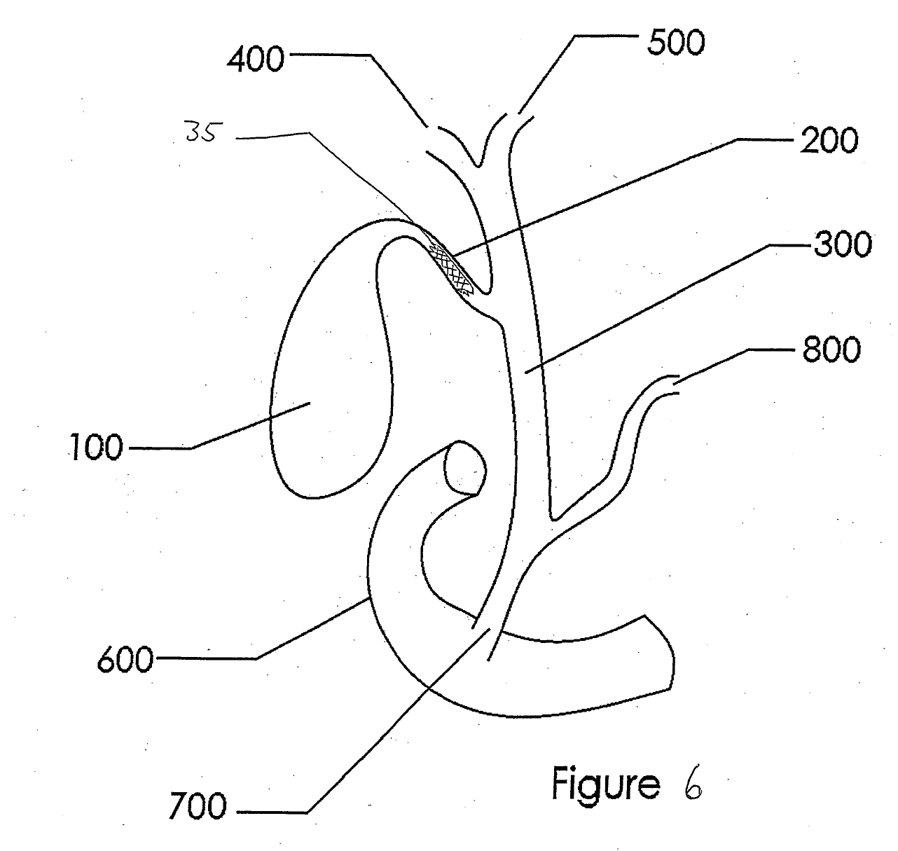

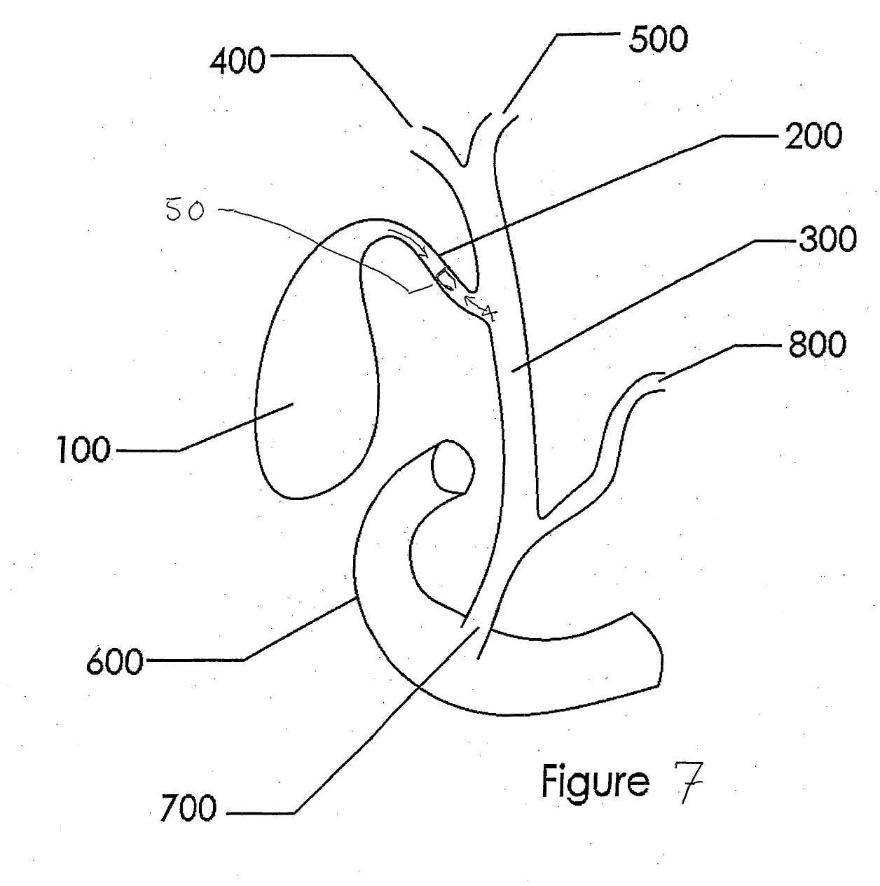

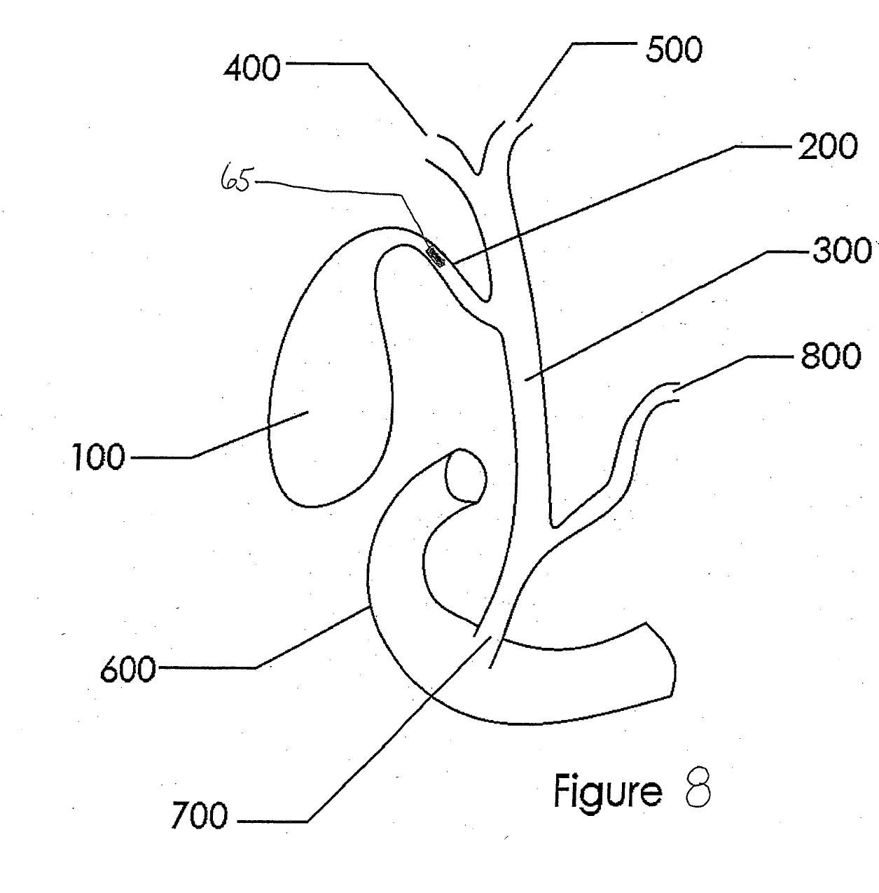

According to yet another aspect of the invention an implant is positioned in the cystic duct 200 either in front of, within, or behind the spiral valves. The device can be an expandable, rigid, or flexible stent 35, coil 35 or tube 35. FIG. 6 is frontal view of a portion of the digestive system and shows an endoluminal stent 35 or tube 35 placed in the cystic duct 200 for restricting, reducing, or modulating flow of bile. FIG. 7 depicts the use of a valve 50 placed within in the cystic duct 200. This valve 50 acts to restrict inflow of bile, but could further reduce flow out of the gallbladder 100. In addition to restricting flow in and out of the gallbladder 100 the valve 50 can be operable to restrict flow in either or both (i.e. 1-way valve) directions. Alternatively, a plug 65 could be placed along the cystic duct 200 stopping all flow as show in FIG. 8. The plug 65 device could be formed in situ using glues or adhesives or be composed of a hydrogel material on an in-situ curing polymer. Such stents 35, tubes 35, coils 35, plugs 65 and valves 50 can be anchored in place or friction fit through expansion. Alternatively a rod or flexible wire like device may simply be threaded through at least a portion of the spiral valves to interfere or limit their and function or the flow of bile. As discussed infra the devices describe herein can be placed temporarily or permanently and can be comprised of biodegradable or bioresorbable materials.

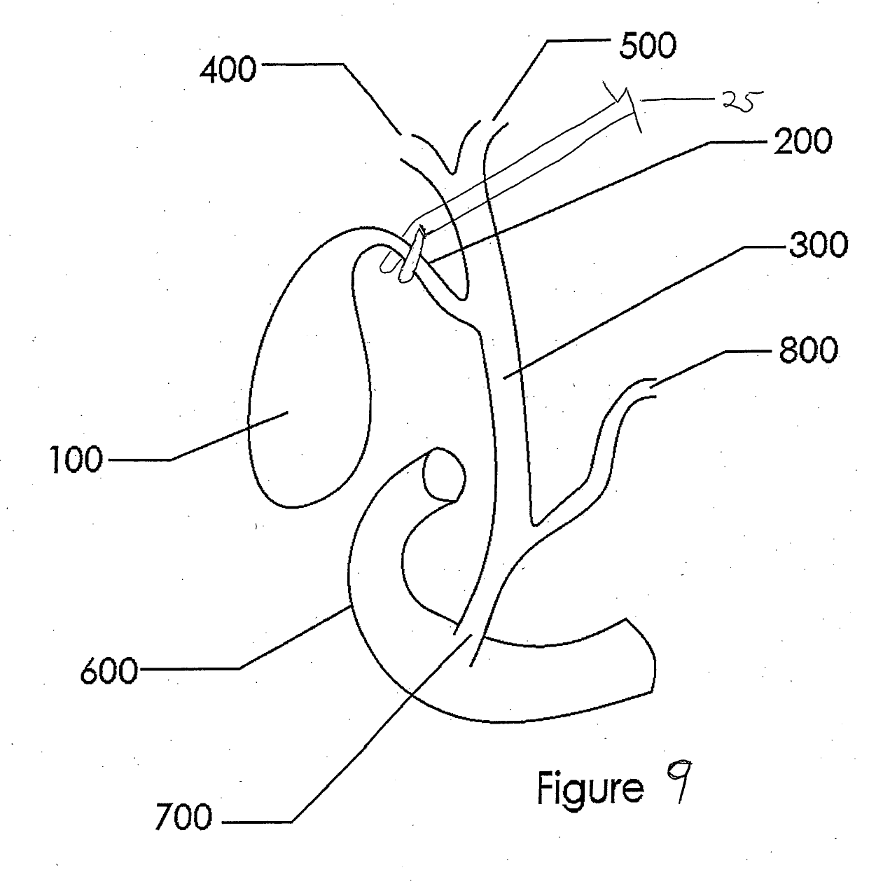

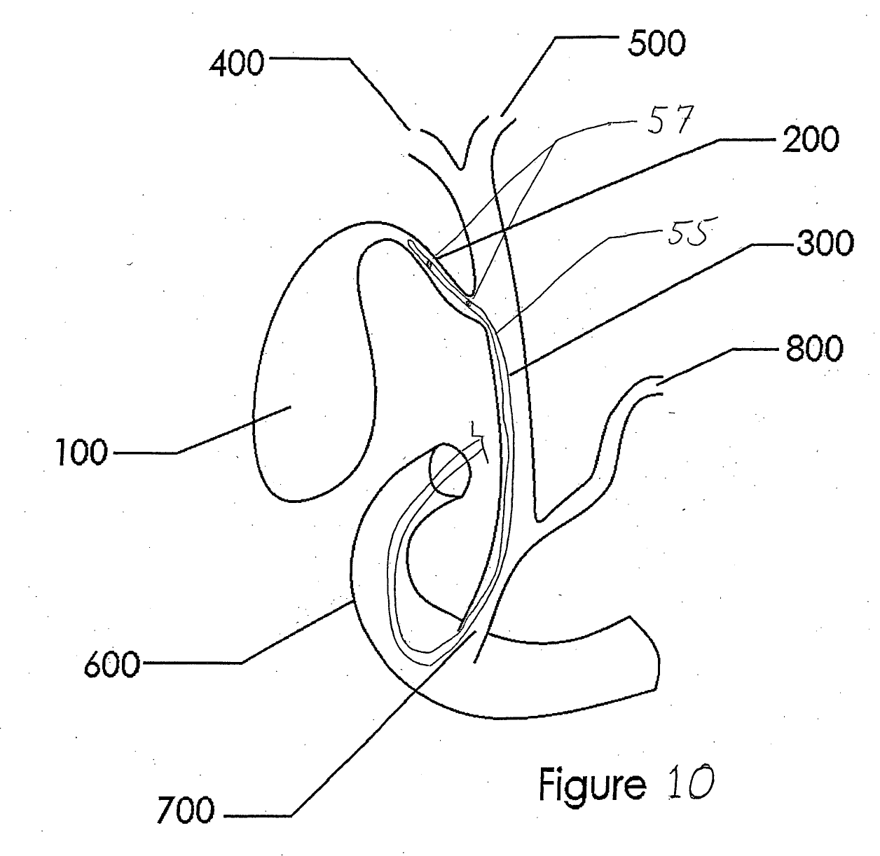

The stricture or stenosis of the cystic duct 200 shown in FIGS. 3 and 4 can alternatively be achieved via the application of energy to or within the duct 200. FIG. 9 shows the application of energy through an RF or other energy grasper 25 placed around cystic duct 200 either through open surgery or laparoscopy. Energy applied to the duct 200 could shrink or damage the tissue surrounding or comprising the duct 200 or specific tissues within the duct 200 such as the spiral valves. In FIG. 10 an energy transducing or radio-frequency catheter 55 is placed endoluminally within the cystic duct 200 to apply heat and cause scarring and/or stricture of the duct 200. Temperature measuring could be employed to optimize the thermal dose delivery. Other forms of energy discussed throughout this disclosure could also be employed.

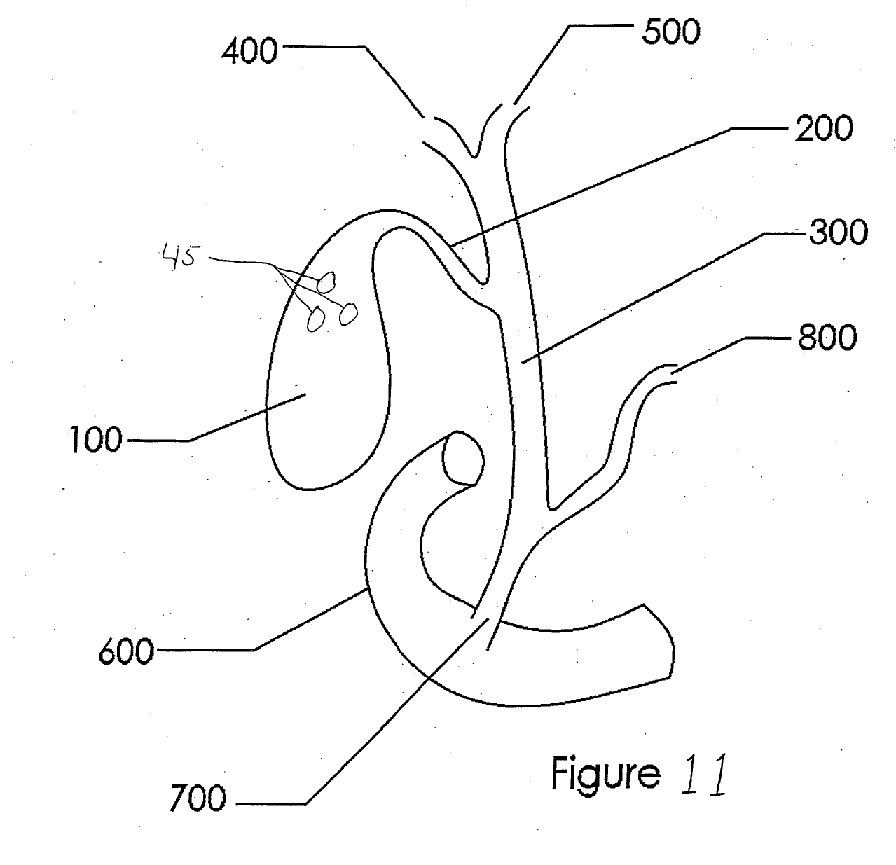

In FIG. 11, a further embodiment of one or more aspects of the invention is shown including one or more artificial gallstones 45 placed within the gallbladder 100. These implants can be delivered either endoluminally or through the gallbladder 100 wall. The artificial gallstones 45 can be drug eluting, expandable, inflatable, biodegradable, and/or radio-opaque. The stones 45 can be sized relative to the cystic duct 200 such that they cannot be passed.

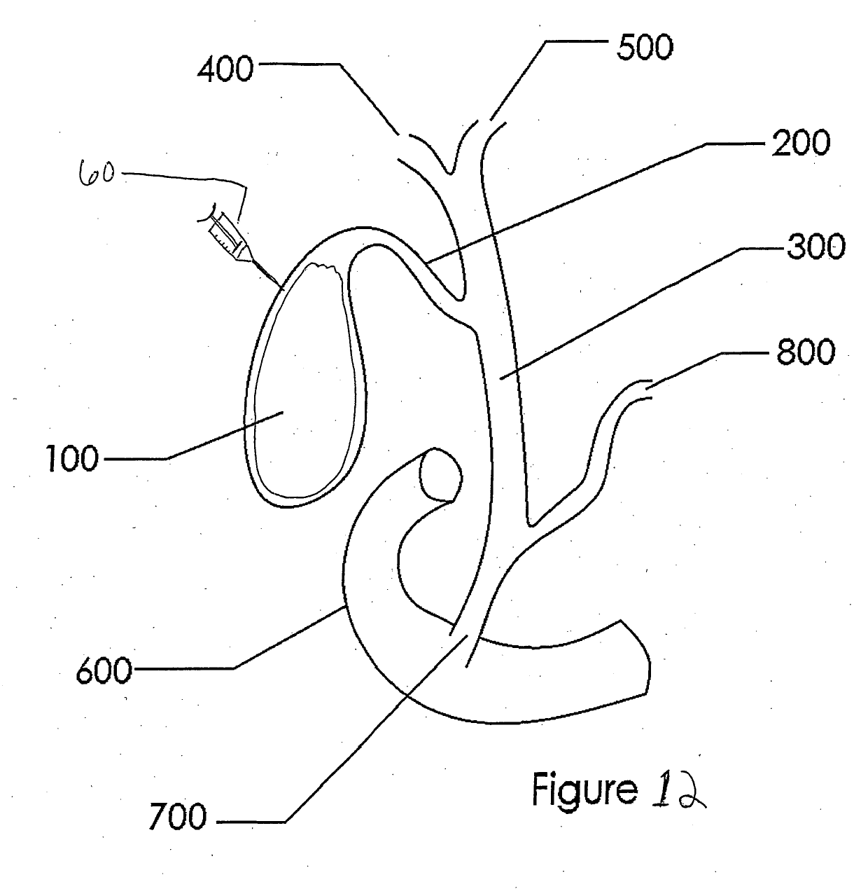

In addition to constricting the exterior of the gallbladder 100 and inserting implants within the organ to alter its function, the walls of the gallbladder 100 can also be treated. FIG. 12 depicts the delivery of flowable substance into the gallbladder 100 wall. The substance could be used to bulk the wall to cause reduction of gallbladder 100 volume, or could be used to cause a reduction in the muscular activity of the wall. Alternatively, shims of wire or plastic could be treaded into the tissue of the organ to interfere with its contraction. Energy could also be applied to various spots along the gallbladder 100 to interfere or damage muscle fibers and nerves to prevent full or efficient contraction of the organ.

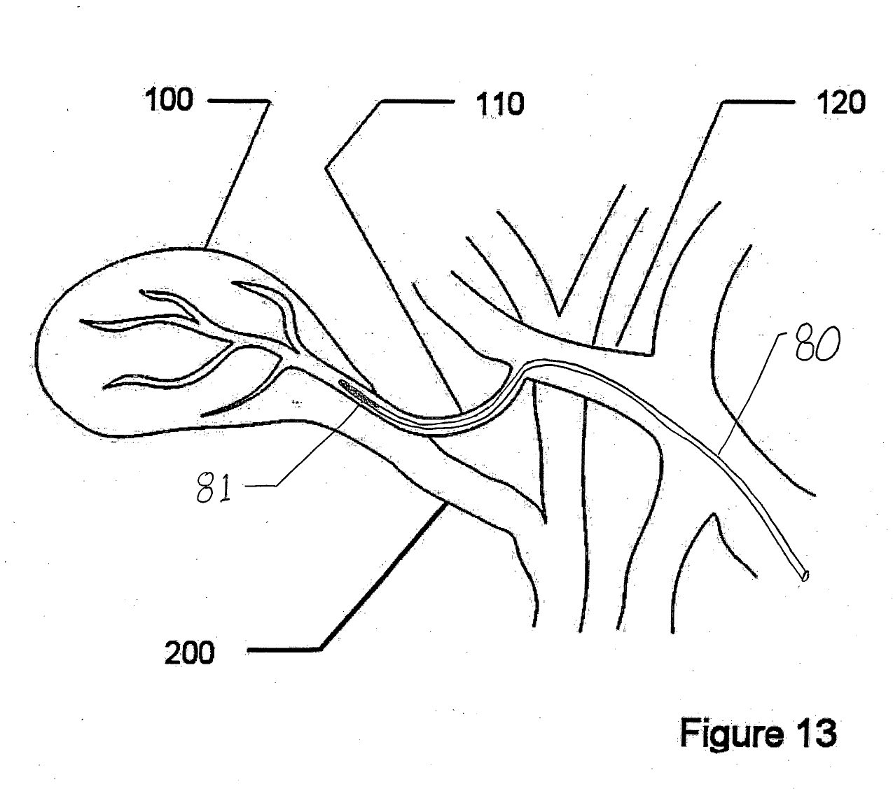

Selective embolisis may also be utilized to alter or limit the function of the gallbladder 100. FIG. 13 depicts a method involving the embolization of the cystic artery 110 to cause a reduction or elimination of flow to at least a portion of the gallbladder 100. Alternatively, distal branches could be selectively embolized.

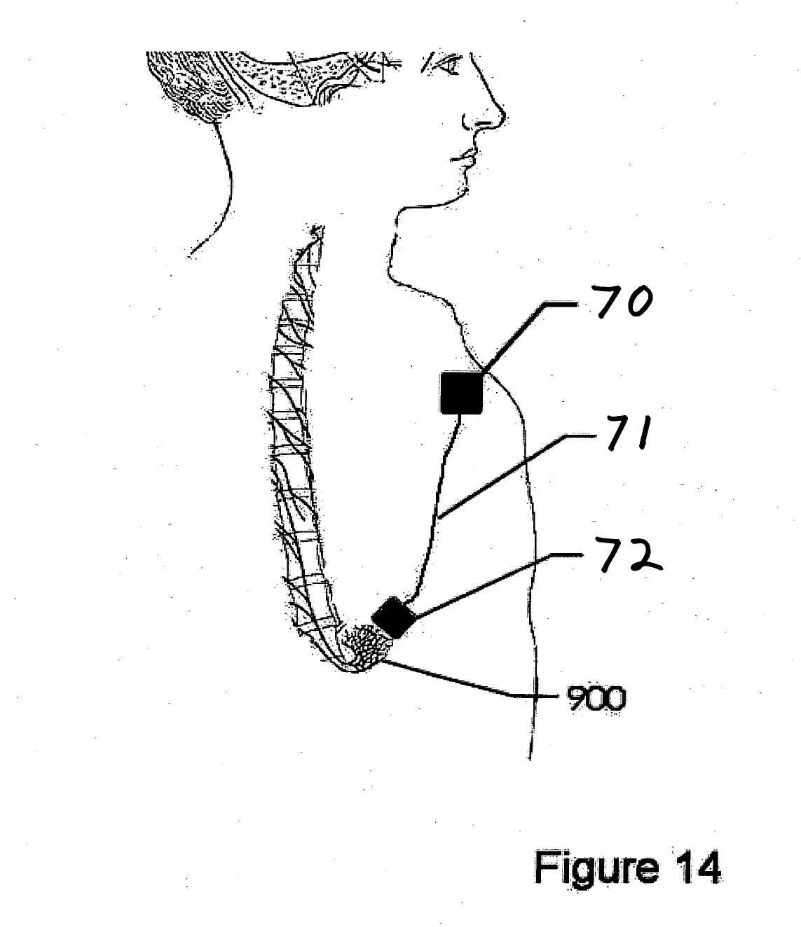

In another embodiment of one or more aspects of the invention pacing to control organ function is utilized. In FIG. 14 electrical pacing of the celiac (caeliac) plexus to cause a change in the activity of the gallbladder 100. Pacer 70,71,72 is shown in the chest wall with a lead wire 71 running to a pacing lead 72 in contact with the celiac plexus. Other sites suitable for placement of pacer leads include:

One or more of the embodiments depicted in FIGS. 1-13 may optionally involve the delivery of an implant or access to a tissue site through endoluminal methods from the digestive tract into the common bile duct then through the cystic duct and into the gallbladder) or through the wall of the gallbladder from either an open or endoscopic surgical approach.

One or more embodiments of the invention as depicted in one or more FIGS. 1-13 comprising devices such as implants, meshes, clips, stents, artificial gall stones, bands coils can be made at least partially of one or more of the following materials: Suitable materials for use with the device of the invention include, but are not limited to, natural or synthetic polymers and co-polymers, plastics, metallic materials and alloys, ceramics, and the like. Further embodiments may comprise materials include any biocompatible material, material of synthetic or natural origin, and material of a resorbable or non-resorbable nature. The devices may also be partially or wholly constructed from material including, but not limited to, autograft, allograft or xenograft; tissue materials including soft tissues, connective tissues, collagen, elastin, and reticulin, demineralized bone matrix and combinations thereof, resorbable materials including polylactide, polyglycolide, tyrosine derived polycarbonate, polyanhydride, polyorthoester, polyphosphazene, calcium phosphate, hydroxyapatite, bioactive glass, collagen, albumin, fibrinogen and combinations thereof; and non-resorbable materials including polyethylene, polylactides, polyglycolic acids, poly(lactide-co-glycolides), polycaprolactones, Polyethylene terephthalate, polyvinyl alcohol (PVA), polyethylene (PE), polyurethane, polypropylene, nylon, polycaprolactone, polycarbonates, polyamides, polyanhydrides, polyamino acids, polyortho esters, polyacetals, polycyanoacrylates, and degradable polyurethanes, polyester, polyvinyl alcohol, polyacrylonitrile, polyamide, polytetrafluorethylene, EPTFE, polyparaphenylene terephthalamide, polyformaldehyde, fluorinated ethylenepropylene co-polymer, polyphenylene oxide, polypropylene cellulose, and combinations thereof. Further examples of non-resorbable materials include carbon-reinforced polymer composites, shape memory alloys, titanium, titanium alloys, cobalt chrome alloys, stainless steel, and combinations thereof.

One or more of the embodiments depicted in FIGS. 1-13 may also incorporate a drug eluting device, seeded device containing paralytics such as botulism toxin, hormones such as CCK, gastrin, CCK blocking compounds, and tissue destroying or cell lysing agents such as alcohol. Alternatively the method depicted in one or more FIGS. 1-13 may involve the step of treating or injecting at least a portion of tissues of the biliary system with the aforementioned chemicals. Such chemicals my also be used to induce a partial neuropathology to a portion of the biliary system. Other suitable chemicals and bioactive agents include, but are not limited to, tissue growth enhancing substances such as growth factors, angiogenic factors, immune system suppressors such as anti-inflammatory agents, antibiotics, living cells, cell-binding proteins and peptides, and the like. Growth factors which enhance cartilage repair are particularly preferred for use as bioactive agents. Examples of suitable growth factors are selected from the group consisting of somatomedins (somatomedin-C), insulin-like growth factors (such as IGF-I and II), fibroblast growth factors (including acidic and basic FGF), bone morphogenic factors (e.g., BMP and BMP2), endothelial cell growth factors, transforming growth factors (TGF alpha and beta), platelet derived growth factors (“PDGF”), hepatocytic growth factors, keratinocyte growth factors, and combinations thereof. Growth factors that function by attracting fibroblasts are preferred, as are growth factors that encourage fibroblast growth, either directly or indirectly by encouraging mesenchymal cell development.

One or more embodiments of the invention as depicted in one or more FIGS. 1-13 can optionally further involve the steps of monitoring the flowrate of bile either into, out of or both through of the cystic duct via direct sensing or through various imaging modalities such as X-ray or fluoroscopic observation. The monitoring could be conducted prior to, during and after the procedure. The monitoring could be used to achieve or determine a selected flow rate, ejection fraction, ejection volume as discussed previously.

One or more embodiments of the invention as depicted in one or more FIGS. 1-13 can optionally further involve a temporary or reversible procedure or involve the use of a biodegradable implant operable not to require explantation at the termination of the treatment.

One or more embodiments of the invention as depicted in one or more FIGS. 1-13 can optionally involve a combined approach utilizing an embodiment depicted in another figure to enhance the overall effect of the treatment. For example, a portion of the gallbladder could be paralyzed via the injection of botulism toxin and then an artificial gall stone could be implanted within the gallbladder. Similarly, a CCK inhibitor eluting patch could be attached to the duodenum and a down regulating valve could be implanted within the cystic duct.

1. A method of treating obesity comprising:
Permanently or temporarily lowering one or more of the following aspects of a subjects gallbladder: motility, ejection fraction, turnover rate, bile flow rate;
wherein said lowered aspect causes the consumption of certain amounts and types of food to result in physical discomfort in the subject.

2. The method in claim 1 further comprising the step of applying energy to a portion of the gallbladder or cystic duct.

3. The method in claim 1 further comprising the step of causing a stricture or stenosis in at least a porting of the gallbladder or cystic duct.

4. The method in claim 1 further comprising the step of inserting an implant within the gallbladder or cystic duct wherein said device at least partially limits the flow of bile or interferes with the contraction of the gallbladder.

5. The method in claim 1 further comprising the step of surgically or chemically altering or damaging the tissue of the gallbladder or cystic duct.

6. The method in claim 1 further comprising the step of causing neuropathic damage or paralyzing a portion of the cystic duct.

7. The method in claim 1 further comprising the step of diminishing the capacity of duodenum to deliver CCK or inhibiting the capacity of the gallbladder to absorb CCK.

8. The method in claim 1 further comprising the step pacing at least a portion of the biliary system.

9. The method in claim 1 further comprising the step of causing neuropathic damage or paralyzing a portion of the gallbladder.

10. The method in claim 1 further comprising the step of monitoring an aspect of the biliary system.

11. The method in claim 1 further comprising the step of lowering the evacuation fraction of the gallbladder to between 80 and 10 percent.

12. The method in claim 1 further comprising the step of placing on the gallbladder a clip-like device operable to constrict or prevent the expansion of at least a portion of the gall bladder.

13. The method in claim 1 further comprising the step of injecting the gallbladder with botulism toxin.

14. The method in claim 1 further comprising the step of inserting one or more artificial gallstones in the gallbladder.

15. The method in claim 1 further comprising the step of implanting a drug or agent eluting patch, stent, or device on or within the gallbladder.

16. The method in claim 1 further comprising the step changing the orientation and shape of the gallbladder and cystic duct by torsion.

17. A method of encouraging a subject to eat a diet limited in fat comprising the steps of:
accessing an internal or external surface of a cystic duct; and causing or allowing the temporary or permanent stricture or stenosis of a cystic duct such that flow through the duct is at least partially restricted.

18. The method in claim 17 further comprising the step of causing at least partial torsion of the cystic duct thereby restricting flow therethrough.

19. A method of modulating gall bladder ejection fraction and motility comprising:
accessing the interior of a gallbladder; and inserting an artificial gallbladder stone wherein said stone is sized not to be passable via the cystic duct.

20. The method in claim 19 wherein the step of accessing the interior of a gallbladder comprises endoluminally expanding the cystic duct sufficient to pass an artificial gallstone and inserting the stone beyond the duct and within the gallbladder

21. A device for encouraging a subject to avoid certain types and amounts of food comprising:
one or more expandable artificial gallstone wherein said gallstone has a smaller volume or shape defining a delivery profile and a second larger volume or profile defining its implanted profile.

22. A device for modulating the function of the biliary system comprising a constrictive mesh adapted for placement about the exterior of at least a portion of the gallbladder and cystic duct wherein said mesh constricts or limit the expansion of at least a portion of the gallbladder and duct.

23. The device in claim 22 wherein said mesh is shaped like a pouch and has a neck opening for placement around the cystic duct.

24. The device in claim 22 wherein said mesh is biodegradable or bioresorbable.

25. The device in claim 22 wherein said mesh is cylindrical with a narrowed center portion operable to prevent slippage off of said gallbladder.
